(19) United States
(12) Patent Application Publication
(10) Pub. No.: US 2005/0172958 A1
(43) Pub. Date: Aug. 11, 2005

(54) Inhalation device and system for the remote monitoring of drug administration

(75) Inventors: Michael S. Singer, Cambridge, MA (US); Bruce D. Levy, West Roxbury, MA (US)

Correspondence Address: FITCH, EVEN, TABIN & FLANNERY, P. O. BOX 65973, WASHINGTON, DC, 20035, US

(73) Assignee: The Brigham and Women's Hospital, Inc.

(21) Appl. No.: 11/037,903

(22) Filed: Aug. 18, 2004

Related U.S. Application Data
(60) Provisional application No. 60/496,408, filed on Aug. 20, 2003.

(51) Int. Cl. A61M 11/00

(52) U.S. Cl. 128/200.23; 128/200.14

(57) ABSTRACT

The present invention is directed to a device for monitoring the usage of inhaled drugs by a patient. The device includes an inhaler, a use sensor, a microprocessor, a wireless transmitter and a battery compartment. These components allow information concerning drug usage to be transmitted to health care personnel that can evaluate the data to determine whether there are changes in drug usage characteristics that are indicative of an impending acute attack. The invention includes not only the device, but also the systems and methods in which the device is employed.

### CROSS REFERENCE TO RELATED APPLICATIONS

The present application claims the benefit of U.S. provisional application No. 60/496,408, filed on Aug. 20, 2003, which is incorporated in its entirety herein by reference.

### FIELD OF THE INVENTION

The present invention is directed to a medical device for administering drug to a patient by inhalation. The device records the time of administration and transmits this information to a remote receiver. The invention also includes systems for monitoring the amount of medication being taken by a patient using the device.

### BACKGROUND OF THE INVENTION

Over twenty million Americans suffer from asthma or chronic obstructive pulmonary disease (COPD). These diseases are characterized by periods of relative normalcy punctuated by acute attacks that may be severe enough to require hospitalization. Typically, an attack is preceded by a progressive increase in a patient's use of “rescue” medication to alleviate respiratory difficulties and a decrease in lung function, as measured by peak expiratory flow rate. These changes usually occur several days or weeks before an attack and can serve as a signal for initiating preemptive treatment. Unfortunately, patients often lack the time or resolve to keep accurate records of drug usage. As a result, they may not become aware that their condition is deteriorating until it is too late to prevent an attack requiring urgent medical attention. Also, many elderly or impaired patients lack the capacity for carefully monitoring changes in drug use patterns.

Many different types of inhalation devices have been developed and used by respiratory patients for delivering a carefully controlled dosage of medication (see, e.g., U.S. Pat. Nos. 6,223,746; and 6,532,955). Some of these devices have microprocessors and sensors for counting the number of doses administered (U.S. Pat. Nos. 6,138,669; and 5,593,390) or have other adaptations to improve delivery characteristics (U.S. Pat. No. 5,477,849). However, the devices continue to rely upon patients to monitor their own drug use patterns. An inhalation device which allowed health care providers to monitor respiratory patients would avoid the problems inherent in self-monitoring and would represent a significant advance in the clinical treatment of these patients.

### SUMMARY OF THE INVENTION

The present invention is based upon the development of an inhalation device which contains a microprocessor for recording drug usage information and a wireless transmitter for sending the information to a remote receiver. Preferably the transmitter also includes the ability to receive information from a remote receiver, i.e., the transmitter is in the form of a transmitter/receiver. The invention includes both the monitoring system and the methods by which the device and system are utilized by patients and health care providers. The various components of the invention can be assembled using methods that are standard in the art of medical devices. Existing systems, e.g. that of iMetricus (see www.imetrikus.com and www.imetrikus.com/prod AW.asp) can also be adapted and modified for use in the invention. Additional guidance regarding patient monitoring and monitoring systems may be found in Tovar et al. (Ann. Pharmacother. 38(1): 126-133 (2004)); Marosi et al. (J. Asthma 38(8): 681-690 (2001)); and Martin et al. (J. Allergy Clin. Immumol. 103(3 Pt. 1): 535-536 (1999)).

In its first aspect, the invention is directed to a device for administering drug to a patient and which contains several components. First, it includes an inhaler for drug administration. The inhaler has an adapter (3) which can form a connection with a separate drug reservoir (9), typically a pressurized canister. The adapter channels the flow of medication from the drug reservoir through a spray inlet (18) and into a flow chamber (11) within the device. In addition to receiving medication from the spray inlet of the adaptor, the flow chamber has a fresh air inlet (23) which permits air to mix with medication during drug delivery. The fresh air inlet may be at the medication module (2) or there may be a separate inlet to the flow chamber located elsewhere (for example, in the housing near the medication module). The inhaler also includes a mouthpiece (7) which is connected to the flow chamber (11) and funnels the mixture of air and medication outside the device to the patient. The inhaler also has a use sensor (8) which is connected to a microprocessor by an electrical circuit and which transmits electrical signals to the microprocessor in response to the passage of medication through the flow chamber (11).

A second component of the device is the microprocessor which, as mentioned above, is connected by an electrical circuit to the use sensor (8) and which, in response to receiving electrical signals from the use sensor, records the time. The microprocessor is also connected to a third component of the device, a wireless transmitter. In response to electrical signals from the microprocessor, the transmitter sends radio frequency waves which may be received by a remote recipient. When a transmitter/receiver is used, the remote recipient, typically a health care worker, can communicate back to the patient. For example, the remote recipient may send a message back to the patient that is shown on the digital display of the inhaler and which indicates that there has been a change in their condition.

The inhalation device also includes a battery compartment which is electrically connected to one or more of the use sensor, microprocessor or wireless transmitter. The battery compartment includes contacts for receiving electrical input from one or more batteries.

In preferred embodiments, the device described above includes a pressurized drug reservoir (9), typically in the form of a canister, which is connected to the adapter (3). Connection may be accomplished by means of an adapter peg (15) having a spray outlet (17). The pressurized canister will typically include a metered dose reservoir (16) which contains a fixed dosage for administration to a patient. The canister should also include a one way valve (14) that opens to allow the pressurized flow of drug through the spray outlet (17) in response to compression of the adapter peg (15).

In another preferred embodiment, the use sensor (8) is in the form of an electrical switch which has both positive and negative electrical contacts (20, 21). The switch may make contact with the drug reservoir (9) by means of a contact rod (19) which closes the switch in response to movement of the drug reservoir.

The inhalation device may optionally include an additional diagnostic component called a peak expiratory flow meter (10) located within the flow chamber (11). The peak expiratory flow meter is electrically connected to the microprocessor and records the flow rate of air blown into the flow chamber (11) by the patient. The flow meter may be in the form of a flow turbine (12) which spins in response to the flow of air and which communicates with the microprocessor by means of a spin sensor (13). Thus, the inhalation device may provide diagnostic information both with respect to drug usage and with respect to lung capacity. Alternatively, the peak expiratory flow meter may be supplied as a separate device having its own microprocessor and transmitter or transmitter/receiver.

In another aspect, the invention is directed to a system for monitoring the drug inhalation characteristics of a patient (see e.g., FIG. 4). The system is made up of any of the devices described above (FIG. 4A) and a remote receiver (FIG. 4B) that receives input from the wireless transmitter to record the time of drug delivery and, preferably, expiratory flow rate information. The system will typically display data on a computer monitor (FIG. 4C) and then may transmit this data by means of the internet to a second computer (FIG. 4D) that is monitored by a health care provider (FIG. 4E). Based upon this information, the health care provider can detect if drug usage patterns change in a manner indicative of an impending attack.

The invention also includes methods of monitoring drug usage characteristics using the system described above. The method will be of particular use to patients with respiratory diseases, such as asthma and chronic obstructive pulmonary disease. Preferably, the device includes the capacity to both send and receive messages, i.e., it allows for bidirectional communication. Patient to doctor communication would occur automatically as described above, but doctor to patient communication can also occur either via pre-set algorithms or customized specific alerts.

### BRIEF DESCRIPTION OF THE DRAWINGS

The device and system of the present invention are illustrated in FIG. 1-4. The main components shown in the drawings are as follows:

1: the complete device including an outer shell housing its various components;

2: medication module with adaptor (3), use sensor (4) and fresh air inlet (23);

3: adapter which serves to connect the medication reservoir (9) to the device;

4: keypad which can be used by a patient to interface with the microprocessor of the device

5: housing for electronics;

6: digital display;

7: mouthpiece which is used to deliver drug from the device to a patient;

8: use sensor for detecting the delivery of a drug dosage, this may be in the form of a switch with contacts (20 and 21) that are connected by a contact rod (19) in response to drug administration;

9: drug or medication reservoir, typically in the form of a pressurized canister;

10: optional peak expiratory flow meter located within the flow chamber (11);

11: flow chamber;

12: flow turbine which may serve as the peak expiratory flow meter (10);

13: spin sensor transmitting impulses from the flow turbine (12) to a microprocessor;

14: one-way valve separating the metered dose reservoir (16) from the rest of the drug reservoir (9);

15: adapter peg which can be inserted into the adapter of the device (3) to connect the drug reservoir (9);

16: metered dose reservoir holding a measured amount of medication for delivery to a patient;

17: spray outlet located on the adapter peg (15) and which provides a passageway for the entry of drug through the spray inlet of the device (18) and into the flow chamber (11);

18: spray inlet located on adaptor (3);

19: contact rod of the use sensor (8);

20: positive electrical contact of use sensor (8);

21: negative electrical contact of use sensor (8);

22: shelf located within the adapter (3) which contacts the adapter peg (15) of the drug reservoir (9) when the drug reservoir is compressed;

23: fresh air inlet; and

24: indicator light indicating device is on and receiving electrical input from batteries.

FIG. 1: FIG. 1 shows the components of the inhalation device. Panel A is a main view showing the assembled device as seen from above. Panel B is an isolated front view of the mouthpiece. Panel C shows the device in a cutaway view as seen from the mouthpiece. The drawing shows an inserted drug canister (9), a use sensor (8), and an adapter (3) with spray inlet (18). Panel D is a cutaway view of the device as seen from the side. The figure shows an inserted canister (9) attached to an adapter (3). Also shown are a flow channel (11) leading to the mouthpiece (7) and containing a flow meter (10). Panel E is an expanded view of the peak expiratory flow meter (10) as seen from the adaptor looking in the direction of the mouthpiece. The peak expiratory flow meter contains two turbines (12) and two spin sensors (13).

FIG. 2: FIG. 2 contains various views of the drug reservoir (9). The reservoir is seen in an oblique view in panel A. This drug reservoir includes a one-way valve (14) and an adapter peg (15) for connecting to the device. Panel B shows a side view of the drug reservoir illustrating the one-way valve (14), a metered dose reservoir (16) and the adapter peg (15). Panel C is an underside view of the canister showing the metered dose reservoir (16) and the adapter peg (15). Panel D is a side view showing the drug reservoir (9) attached by its adapter peg (15) to the adapter (3). Also shown are the spray inlet of the adapter (18), the one-way valve of the canister (14), the metered dose reservoir (16), and the use sensor (8).

FIG. 3: FIG. 3 contains expanded views of the use sensor and adapter. Panel A shows the contact rod (19) of the use sensor (8) making contact with the medication drug reservoir (9). The adapter peg is shown inserted into the adapter along with both the spray outlet of the drug reservoir (17) and the spray inlet (18) leading to the flow chamber (11). The panel shows the contact rod in both an open (left) and closed position. Panel B is an expanded view of the use sensor (8) showing the contact rod (19) in an open position (left, constituting an open switch) and a closed position (right) in which it connects both the positive and negative electrical contacts (20 and 21) to close the switch. Panel C of the figure shows expanded views of the adapter (3). The left side of Panel C shows a frontal view of the adapter and the spray inlet (9). On the right is a side view showing a small shelf (22) located within the adapter. Arrows show the direction of medication flow.

FIG. 4: FIG. 4 shows a complete system for monitoring drug administration by a patient. The patient uses the inhalation device described above (A) which delivers information concerning drug usage to a communications facility (B). This information is displayed on a computer screen (C) and transmitted via the internet to a second computer (D) which is monitored by a health care provider (E).

### DETAILED DESCRIPTION OF THE INVENTION

The present invention is directed to a device for monitoring the usage of inhaled drugs by a patient. This is accomplished by using a drug inhaler that is equipped with a use sensor, a microprocessor and a wireless transmitter or, preferably transmitter/receiver. The main advantage of this device is that drug usage characteristics are sent to health care personnel for evaluation, thereby avoiding the problems associated with patients monitoring their own changes in drug use patterns. The device, when used in a preferred embodiment, also allows health care personnel to send a message back to the patient, for example, if drug usage characteristics suggest that a respiratory attack is imminent.

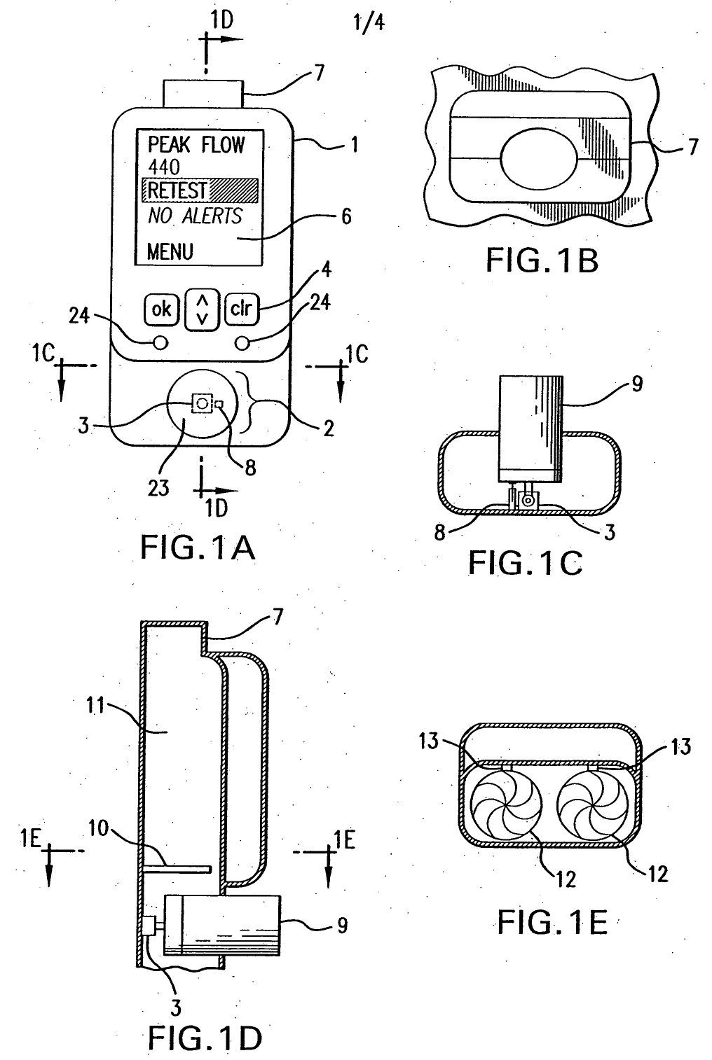

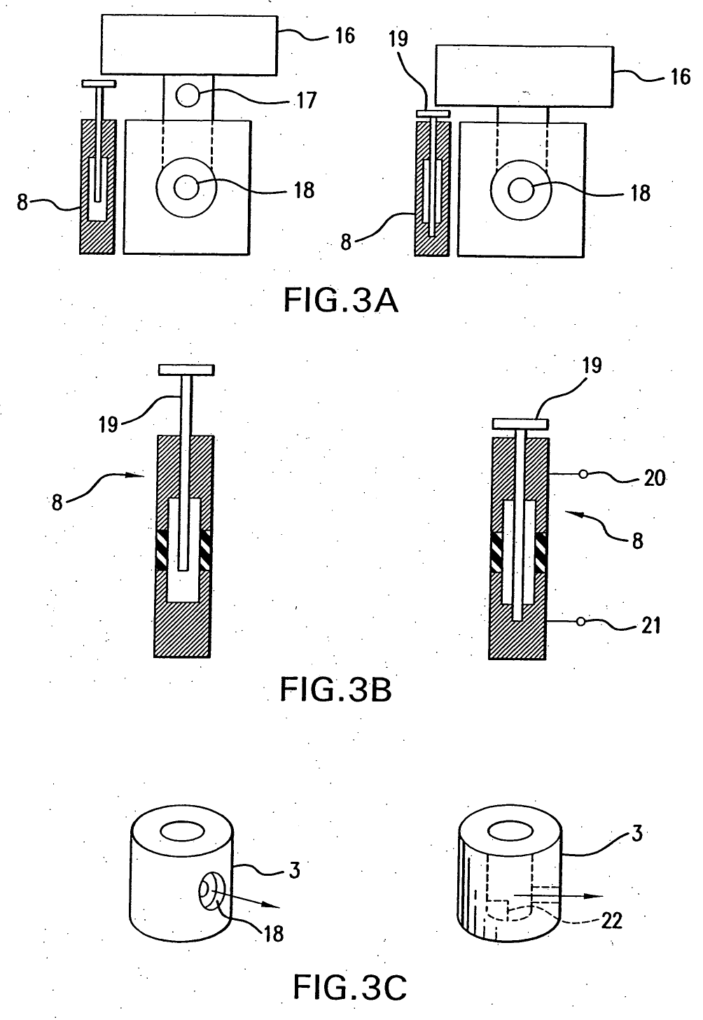

The general characteristics of the device are shown in FIGS. 1-3. The device itself (1) may have a housing constructed of any hard, durable material, such as plastic or metal. It includes one or more medication modules (2) which contain both an adapter (3) for connecting to a drug reservoir (9) and a sensor (8) for detecting when drug is delivered. The canisters which typically serve as drug reservoirs for use in connection with the invention should generally be coated on their inner surface with an inert polymer and should be similar to the canisters described in U.S. Pat. Nos. 6,223,746 and 6,532,955. The use sensor (8) may be essentially a switch as shown in FIG. 3, panels A and B. The main characteristic of the sensor is that it should close an electrical circuit when drug is delivered and, as a result, send a signal to the microprocessor of the device using standard electrical circuitry such as that described in U.S. Pat. No. 6,138,669. The basic switch design in FIG. 3 has a contact rod (19) which is mechanically depressed in response to the downward movement of the drug reservoir. The contact rod should then spring back to its original position opening the circuit when the drug reservoir is retracted. If desired mechanisms, may be included for sensing either electrical or mechanical error/failure. For example, two separate contact rods may be present to allow the microprocessor to determine if one has become jammed in the open or closed position.

The downward movement of the drug reservoir by the patient also has the effect of closing the one-way valve (14) in the drug reservoir (9), thereby limiting the escape of further medication and propellant from the metered dose reservoir (16). Other types of sensor design may also be used in connection with the present invention.

The microprocessor used in the device is also of a standard type and may be incorporated as described, for example, in U.S. Pat. Nos. 6,138,669 and 5,593,390. Its main purpose is to record the clock time of each electrical circuit closure signaled by the use transmitter (8) and to transmit this information by means of a standard digital interface to a wireless transmitter or transmitter/receiver. The basic circuitry and transmitter devices described in U.S. Pat. No. 6,014,429 may be used in connection with the present invention. The wireless transmitter should send the clock times in the form of digital information to a remote receiver, e.g., a computer server. The server can then send the received information via the internet to health care providers.

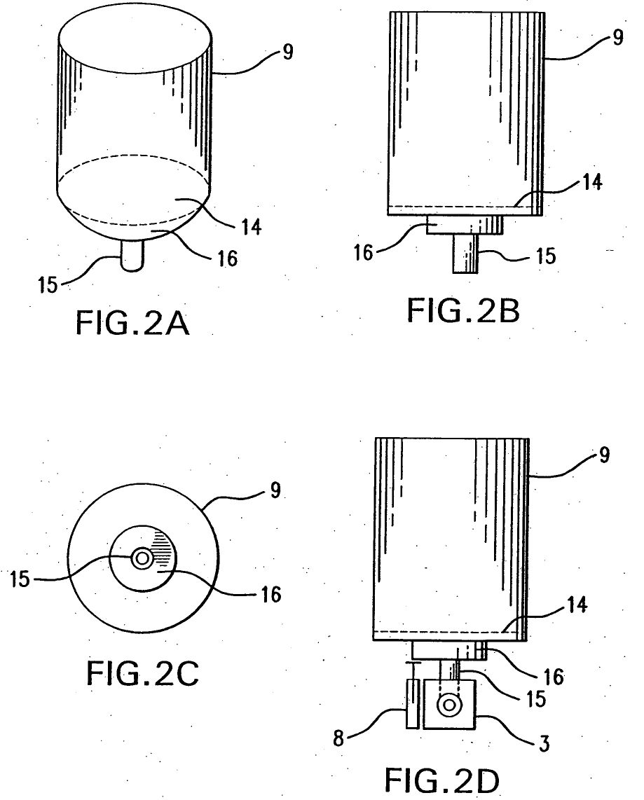

The characteristics of the drug reservoir used in connection with the present invention are shown in FIG. 2 (see also, U.S. Pat. No. 6,223,746). Its main features are the presence of a one-way valve which is ordinarily open, but which closes upon compression of the drug reservoir (9) after connection to the adapter of the device (3). Closure of the valve separates the main body of the canister from the metered dose reservoir (16) which contains the correct dosage of drug for administration to a patient. Compression of the drug reservoir (9) also serves to release drug through a spray outlet (17) located on the side of the adapter peg (15) and into a corresponding spray inlet (18) on the adapter (3).

The spray inlet releases drug into the flow chamber (11) in the device which also has an opening allowing fresh air to enter (23). As shown in FIG. 1, the fresh air opening is located at and is part of the medication module. However, it can also be a separate small opening located elsewhere in the housing. The fresh air mixes with drug and is then inhaled by a patient through the mouthpiece (7). Preferably, there is also a peak expiratory flow meter (10) located within the flow chamber (11) which detects the rate at which the patient can expire air from lungs with maximal effort. In one preferred design, the flow meter is in the form of a flow turbine (12) which signals the microprocessor through a spin sensor (13).

The device should also contain a compartment for batteries with standard contacts that can be used to supply the device with electricity. Any type of standard portable battery is suitable for use with the present invention.

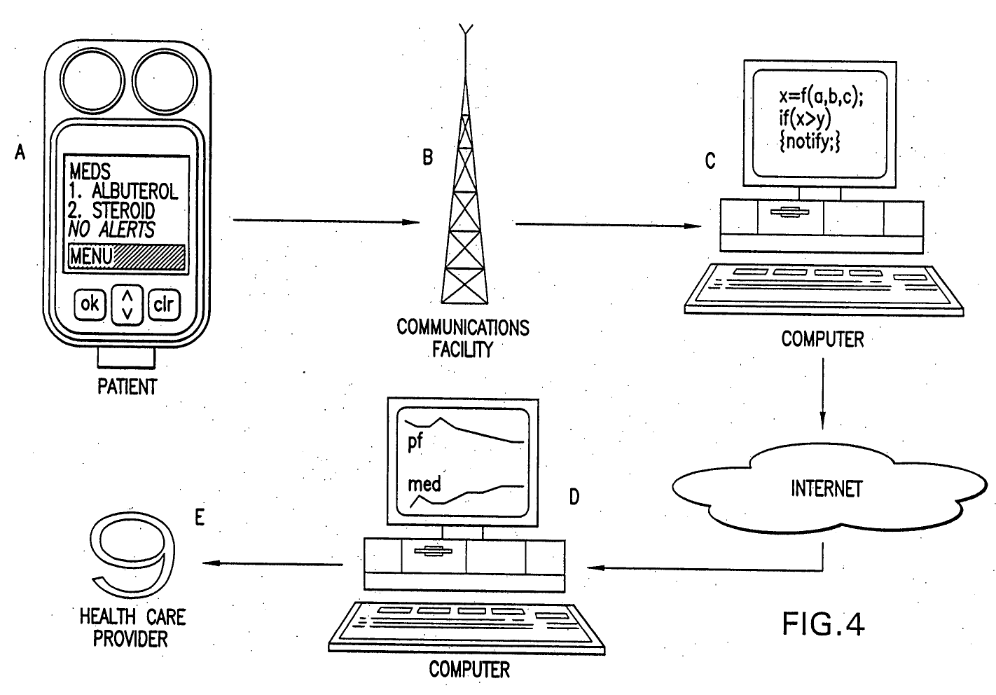

FIG. 4 shows a full system that can be used for monitoring drug administration by a patient. The procedure begins with the device described above (FIG. 4A) sending information regarding times of drug administration or peak expiratory flow to a remote communication facility (FIG. 4B) by means of the wireless transmitter. The communication facility then relays the relevant information to a computer (FIG. 4C) which displays the results. This may then be relayed via the internet to a second computer (FIG. 4D) which is monitored by a health care provider (FIG. 4E). An increase in drug usage and/or a decrease in flow rate is an indication that a patient is likely to soon have an acute attack. A health care provider, faced with this information, would contact the patient to initiate preemptive treatment. This system is likely to be of particular value to patients taking medication by inhalation including respiratory patients experiencing episodic exacerbations. Examples of conditions that would be suitable for monitoring include asthma or chronic obstructive pulmonary disease, cystic fibrosis, non-cystic fibrosis bronchiectasis, forms of interstitial lung disease, reactive airways disease, occupational lung disease, and patients having fluctuations in congestive heart failure control. Because of the frequency of lung involvement, these devices will also be useful in the treatment and monitoring of patients after lung (or other solid organ) transplant or bone marrow transplant. The broad functionality of the device will also make it useful in the close monitoring of medication delivery and lung function that is necessary for clinical research trials. Importantly, the device and system shifts the responsibility for drug monitoring from the patient to trained individuals better able to interpret data and more likely to be conscientious in detecting drug usage changes. Obviously, other designs for systems may be used equally well to that shown in FIG. 4. Again, the main objective is to provide drug usage information directly to health care personnel.

All references cited herein are fully incorporated by reference. Having now fully described the invention, it will be understood by one of skill in the art that the invention may be performed within a wide and equivalent range of conditions, parameters, and the like, without affecting the spirit or scope of the invention or any embodiment thereof.

1. A device for administering drug to a patient by inhalation, comprising:
(a) an inhaler comprising:
(i) an adapter for connecting to a drug reservoir and which channels the flow of medication from said drug reservoir into a flow chamber;
(ii) said flow chamber which receives medication from said adapter and which additionally has a fresh air inlet;
(iii) a mouthpiece connected to said flow chamber and which is capable of delivering drug outside said device;
(b) a use sensor connected to a microprocessor by an electrical circuit and which transmits an electrical signal to said microprocessor in response to drug administration;
(c) a microprocessor connected to said use sensor by said electrical circuit and which, in response to said electrical signal, records the time of said signal and which is electrically connected to a wireless transmission device;
(d) a wireless transmission device, connected to said microprocessor and which, in response to electrical signals from said microprocessor, transmits radio frequency waves; and
(e) a battery compartment which is electrically connected to one or more of said use sensor, microprocessor and wireless transmission device and which has contacts for receiving electrical input from one or more batteries.

2. The device of claim 1, further comprising a pressurized drug reservoir connected to said adapter by means of an adapter peg containing a spray outlet.

3. The device of claim 2, wherein said drug reservoir is in the form of a canister with an outlet valve that opens to allow the flow of drug through said spray outlet in response to the compression of said adapter peg.

4. The device of claim 1, wherein said use sensor is in the form of an electrical switch which makes contact with said drug reservoir and which is closed in response to movement of said drug reservoir.

5. The device of claim 1, further comprising a peak expiratory flow meter located within said flow chamber, wherein said flow meter is electrically connected to said microprocessor and records the patient's peak expiratory flow rate.

6. The device of claim 5, wherein said flow sensor comprises a flow turbine which spins in response to medication flow in said flow chamber, and which is electrically connected to said microprocessor.

7. The device of claim 6, further comprising a spin sensor which connects with said flow turbine and transmits electrical signals regarding medication flow to said microprocessor.

8. A system for monitoring the drug inhalation characteristics of a patient, comprising:
(a) the device of any one of claims 1-7; and
(b) a remote receiver that receives input from said wireless transmitter of said device.

9. A method for monitoring the drug of a patient, comprising recording the drug inhalation characteristics of said patient using the system of claim 8.

10. The method of claim 9, wherein said patient has a respiratory disease.

11. The method of claim 9, wherein said patient has asthma or chronic obstructive pulmonary disease.

12. The method of claim 9, wherein said patient has cystic fibrosis, non-cystic fibrosis bronchiectasis, forms of interstitial lung disease, reactive airways disease, occupational lung disease, or congestive heart failure.

13. The method of claim 9, wherein said patient has received a solid organ transplant or bone marrow transplant.

14. The method of claim 9, wherein said patient is the subject of a clinical research trial.
